(19) United States
(12) Patent Application Publication
(10) Pub. No.: US 2024/0008867 A1
(43) Pub. Date: Jan. 11, 2024

(54) SUTURE PASSER DEVICES AND METHODS

(71) Applicant: Kevin Ray Myers, Plano, TX (US)

(72) Inventor: Kevin Ray Myers, Plano, TX (US)

(21) Appl. No.: 18/172,649

(22) Filed: Feb. 22, 2023

Related U.S. Application Data
(60) Provisional application No. 63/368,134, filed on Jul. 11, 2022.

(51) Int. Cl. A61B 17/04; A61B 17/062

(52) U.S. Cl. CPC A61B 17/0469; A61B 17/0625

(57) ABSTRACT

A suture passer device to pass a suture through a tissue may include a handle, an elongate body, and a suturing head. The suturing head may include a first jaw member and a second jaw member. The first jaw member may include a proximal part and a distal part movable along a length of the proximal part. The distal part may include an opening at a distal end that can capture a length of a suture from a needle. The second jaw member may be pivotally coupled to the first jaw member to form a distal-facing mouth operable to clamp a suture site of the tissue in a closed configuration. The second jaw member can guide the needle toward the opening of the distal part of the first jaw member. A distal portion of the second jaw member may include a movable ramp.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

### Field

This disclosure generally relates to devices and methods for passing a suture through tissue.

### Description of the Related Art

There exist several devices and methods to suture soft tissue in connection with surgical procedures. In such surgical procedures, suturing and associated manipulation is typically performed in confined areas which are not easily accessible. Current devices and methods do not provide for easy manipulation of the suturing devices. Moreover, most current devices are limited in their implementation to areas which are visually accessible. Therefore, there is a need for improved suture devices and methods to address these and other drawbacks of existing solutions.

### SUMMARY

The embodiments disclosed herein each have several aspects, of which no single one is solely responsible for the disclosure's desirable attributes. Without limiting the scope of this disclosure, the prominent features are briefly discussed herein. After considering this discussion, and particularly after reading the section entitled “Detailed Description,” one will understand how the features of the embodiments described herein provide advantages over existing systems, devices, and methods relating to suture passer devices and methods.

The following disclosure describes non-limiting examples of some embodiments. For instance, other embodiments of the disclosed devices and methods may or may not include the features described herein. Moreover, disclosed advantages and benefits may apply only to certain embodiments and should not be used to limit the disclosure.

Suture passer devices and methods are described herein.

In one aspect of the present disclosure, a suture passer device configured to pass a suture through a tissue may include a handle, an elongate body extending from the handle, and a suturing head at a distal end of the elongate body. In some embodiments, the suture passer device may be handheld.

In some embodiments, the suturing head may include a first jaw member and a second jaw member.

The first jaw member may include a proximal part and a distal part. The distal part may be movably coupled to the proximal part. The distal part may be movable along a length of the proximal part. The distal part may include an opening at a distal end thereof. The opening may be configured to capture a length of a suture from a needle.

The second jaw member may be pivotally coupled to the first jaw member to form a distal-facing mouth. The distal-facing mouth may be configured to clamp a suture site of the tissue in a closed configuration. The second jaw member may be configured to allow movement of the needle between a retracted position and an extended position. The second jaw member may have a distal end configured to guide the needle toward the opening of the distal part of the first jaw member.

In some embodiments, the distal end of the second jaw member may include a ramp. The ramp may be configured to guide the needle toward the opening of the distal part of the first jaw member.

In some embodiments, an angle of the ramp may be adjustable.

In some embodiments, the suture passer device may include a needle configured to selectively engage a length of a suture. In some embodiments, the needle may be configured to extend from the distal end of the second jaw member from a retracted position. In some embodiments, the needle may be configured to selectively engage a length of the suture in a recess thereof. In some embodiments, the needle may be configured to penetrate the tissue. In some embodiments, the needle may be configured to extend toward the opening of the distal part of the first jaw member to an extended position. In some embodiments, the needle may be configured to, in the extended position, release a length of the suture to the opening of the distal part of the first jaw member. In some embodiments, the needle may be configured to retract to a proximal end of the second jaw member to the retracted position.

In some embodiments, the recess of the needle may be a slot, wedge or groove.

In some embodiments, the recess of the needle may include a retainer region configured to hold a length of the suture as the needle extends from the retracted position to the extended position. In some embodiments, the retainer region may include a lateral cut-out region of the needle.

In some embodiments, the second jaw member may include a channel so that the movement of the needle between the retracted position and the extended position is along a length of the channel.

In some embodiments, the suture passer device may include a control mechanism configured to control the movement of the needle between the retracted position and the extended position. In some embodiments, the control mechanism may lock the needle in the retracted position when the distal-facing mouth of the suturing head is in an open configuration.

In some embodiments, the first jaw member may be configured to pivot relative to the elongate body. In some embodiments, the suture passer device may include a control mechanism for controlling an angle of pivot of the first jaw member relative to the elongate body. In some embodiments, the first jaw member may be configured to pivot in a range of 0 degrees to 90 degrees relative to the elongate body.

In some embodiments, the second jaw member may be configured to pivot relative to the elongate body. In some embodiments, the suture passer device may include a control mechanism for controlling an angle of pivot of the second jaw member relative to the elongate body. In some embodiments, the second jaw member may be configured to pivot in a range of 0 degrees to 90 degrees relative to the elongate body.

In some embodiments, the second jaw member may be curve-shaped with a given radius of curvature.

In some embodiments, the second jaw member may include a recess at the distal end to selectively engage a length of the suture. In some embodiments, the recess of the second jaw member may be a slot, wedge or groove.

In some embodiments, the second jaw member may include a first loading region configured to hold a length of the suture. In some embodiments, the second jaw member may include a second loading region configured to hold at least one additional length of the suture.

In some embodiments, the ramp at the distal end of the second jaw member may include a recess to selectively engage a length of the suture. In some embodiments, the recess of the ramp may be a slot, wedge or groove.

In some embodiments, the ramp may be semi-circular.

In some embodiments, the tissue may be hip tissue, rotator cuff tissue, and/or meniscus tissue.

In some embodiments, the device may be handheld.

In some embodiments, the opening of the first jaw member may be configured to allow passage of the needle.

In some embodiments, the opening of the first jaw member may include a suture capture member configured to capture a length of the suture from the needle. In some embodiments, the suture capture member may be configured to receive the needle. In some embodiments, the suture capture member may include a spring member, a proximal fixed end and a free distal end. In some embodiments, the suture capture member may be spring loaded by the needle in the extended position, where retraction of the needle may release the suture capture member to spring back and may capture a length of the suture within the suture capture member.

In some embodiments, the opening of the first jaw member may be configured to be a target guide for the needle. In some embodiments, the opening of the first jaw member may be configured to receive the needle.

In some embodiments, the distal part of the first jaw member may be movable in a linear direction along a length of the proximal part.

In some embodiments, the distal part of the first jaw member may be movable in an angular direction relative to a length of the proximal part. In some embodiments, the angular movement of the distal part of the first jaw member may be in a range of 0 degrees to 20 degrees in either direction relative to a length of the proximal part.

In some embodiments, the distal part of the first jaw member may be attached to the proximal part by a pivot joint.

In some embodiments, the distal part of the first jaw member may be configured to be movable in a closed configuration of the distal-facing mouth. In some embodiments, the distal part of the first jaw member may be configured to be movable in an open configuration of the distal-facing mouth.

In some embodiments, the distal part of the first jaw member may be movable by a click wheel. In some embodiments, the click wheel may be arranged at a proximal end of the elongate body.

In some embodiments, the distal part of the first jaw member may be movable by a spring. In some embodiments, release or compression of the spring may be guided by a lever. In some embodiments, the lever may be arranged at a proximal end of the elongate body.

In some embodiments, the angle of the ramp may be adjustable by a lever. In some embodiments, the angle of the ramp may be adjustable by a spring.

In some embodiments, the distal part of the first jaw member may be coupled to the ramp of the second jaw member such that a movement of the distal part of the first jaw member may drive an adjustment of the angle of the ramp. In some embodiments, an extension movement of the distal part of the first jaw member may decrease the angle of the ramp. In some embodiments, a retraction movement of the distal part of the first jaw member may increase the angle of the ramp.

In some embodiments, the distal part of the first jaw member may be configured to be movable and drivably adjust the angle of the ramp in a closed configuration of the distal-facing mouth. In some embodiments, the distal part of the first jaw member may be configured to be movable and drivably adjust the angle of the ramp in an open configuration of the distal-facing mouth.

In some embodiments, the suture may include Nitinol.

In some embodiments, the suturing head may be configured to be removable from the elongate body.

In some embodiments, the elongate body may include a housing to house the needle. In some embodiments, the housing may include a cannula extending along a length of the elongate body.

In another aspect of the present disclosure, a method of passing a suture through tissue may include providing a suture passer device configured to pass a suture through a tissue. The method may include loading a length of the suture into a second jaw member of the suture passer device. The method may include directing the suture passer device with a distal-facing mouth in an open configuration to a site to be sutured.

The method may include adjusting a position of a first jaw member of the suture passer device and a position of the second jaw member so that the distal-facing mouth is in a closed configuration, clamping the tissue to be sutured.

The method may include moving a movable distal part of the first jaw member along a length of a proximal part of the first jaw member to change a position of an opening at a distal end of the first jaw member. The distal-facing mouth may be in the closed configuration. In some embodiments, the distal-facing mouth may be in an open configuration.

The method may include extending a needle of the suture passer device from a retracted position. The needle may be selectively engaged with a length of the suture.

The method may include guiding the needle toward the opening at the distal end of the first jaw member. The method may include pushing the needle through the tissue to be sutured. The needle may guide a length of the suture through the tissue toward the opening at the distal end of the first jaw member. The method may include extending the needle to the extended position. The method may include capturing a length of the suture at the opening at the distal end of the first jaw member. The method may include releasing a length of the suture from the needle to the opening at the distal end of the first jaw member. The method may include retracting the needle to the retracted position. The method may include adjusting a position of the first jaw member and a position of the second jaw member so that the distal-facing mouth is in an open configuration. The method may include directing the suture passer device away from the suture site, wherein the opening at the distal end of the first jaw member is selectively engaged to a length of the suture.

In some embodiments, the method may include adjusting an angle of a ramp arranged at a distal end of the second jaw member to guide the needle toward the opening at the distal end of the first jaw member.

In some embodiments, a method of passing a suture through tissue may include engaging a tissue with a suture device including a lower jaw member and an upper jaw member. The upper jaw member may be pivotable relative to the lower jaw member and may include a proximal part and a movable distal part.

The method may include translating the movable distal part relative to the proximal part along a longitudinal direction of the upper jaw member. The method may include directing a suture through an opening in the upper jaw member to close the tissue.

In some embodiments, the method may include directing the suture along the lower jaw member to engage a ramp. The ramp may direct the suture into the tissue.

In some embodiments, the method may include adjusting an angle of the ramp relative to the lower jaw member.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings. In the following detailed description, reference is made to the accompanying drawings, which form a part of the disclosure hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawings, may be arranged, substituted, combined, and designed in a wide variety of different configurations, all of which are explicitly contemplated and made part of this disclosure.

FIG. 1 is a schematic side view of an example of a suture passer device.

FIG. 2 is an enlarged schematic side view of the suturing head of the suture passer device of FIG. 1.

FIG. 3 is an enlarged schematic top view of an example of a first jaw member of a suturing head of the suture passer device of FIG. 1, showing a proximal part and a linear direction of movement of a movable distal part along a length of the proximal part.

FIG. 4 is an enlarged schematic top view of an example of a first jaw member of a suturing head of the suture passer device of FIG. 1, showing a proximal part and an angular direction of movement of a movable distal part along the proximal part.

FIGS. 5A and 5B are enlarged schematic side views of an example a suturing head of the suture passer device of FIG. 1, showing a distal-facing mouth formed by the first jaw member and the second jaw member in an open configuration and closed configuration, respectively.

FIGS. 6A-6D are enlarged schematic side views of an example suturing head of the suture passer device of FIG. 1, showing an operation thereof.

FIG. 7 is a schematic side view of another example of a suture passer device.

FIG. 8 is an enlarged schematic side view of an example suturing head of the suture passer device of FIG. 7.

FIG. 9 is an enlarged schematic top view of an example of a first jaw member of a suturing head of the suture passer device of FIG. 7, showing a proximal part and a linear direction of movement of a movable distal part along a length of the proximal part.

FIG. 10 is an enlarged schematic top view of an example of a first jaw member of a suturing head of the suture passer device of FIG. 7, showing a proximal part and an angular direction of movement of a movable distal part along the proximal part, according to some embodiments.

FIGS. 11A and 11B are enlarged schematic side views of an example suturing head of the suture passer device of FIG. 7, showing a distal-facing mouth formed by the first jaw member and the second jaw member in an open configuration and closed configuration, respectively.

FIG. 12 is an enlarged schematic side view of an example of a second jaw member of a suturing head of the suture passer device of FIG. 7, showing a ramp and a direction of movement of the ramp relative to the second jaw member.

FIGS. 13A and 13B are enlarged schematic side views of an example suturing head of the suture passer device of FIG. 7, showing changes in an angle of the ramp relative to the second jaw member due to extension and/or retraction of the distal movable part of the first jaw member relative to the proximal part of the first jaw member.

FIGS. 14A-14C are enlarged schematic side views of an embodiment of the suturing head of the suture passer device of FIG. 7, showing an operation thereof.

FIG. 15 is a block diagram of an example method of passing a suture through a tissue using the suture passer device described herein

### DETAILED DESCRIPTION

The following detailed description is directed to certain specific embodiments of suture passer devices and methods. In this description, reference is made to the drawings wherein like parts or steps may be designated with like numerals throughout for clarity. Reference in this specification to “one embodiment,” “an embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments. The embodiments of the invention, examples of which are illustrated in the accompanying drawings, are set forth in detail below. Wherever possible, the same reference numbers are used throughout the drawings to refer to the same or like parts.

Throughout this specification, reference is made to a tissue to be sutured. The term tissue is to be fairly construed and encompasses all tissues of a body that can be sutured. Such tissues may include tissues which are torn, cut or punctured, where trauma causes a contusion, or any other conditions or imperfections in the tissue of a patient or any tissues that may otherwise that may benefit from suturing. A tissue to be sutured can include any tissue, including, but not limited to, separated skin layers and/or separated muscle fibers, where a gap may or may not be present, where suturing may bring different layers, fibers, etc. of the tissue closer to each other. Suturing tissues may or may not be conducted as a part of a surgical procedure. Examples of suturing include, but are not limited to, suturing abdominal wounds or other large or incisional wounds, either as a result of surgery, trauma, sternotomies, fasciotomies, or other conditions, dehisced wounds, acute wounds, chronic wounds, subacute and dehisced wounds, traumatic wounds, flaps and skin grafts, lacerations, abrasions, contusions, bums, diabetic ulcers, pressure ulcers, stoma, surgical wounds, trauma and venous ulcers or the like.

In some embodiments, the tissue to be sutured may include, but not be limited to, hip tissue, rotator cuff tissue, and/or meniscus tissue.

Throughout this specification, reference is made to a suture. The term suture is to be fairly construed and encompasses all types of sutures. Suturing materials may include absorbable and/or non-absorbable materials. Such materials may include, but are not limited to, Polyglycolic Acid, Polyglactin 910, Catgut, Poliglecaprone 25, Polydioxanone, Polypropylene, Nylon (polyamide), Polyester, Polyvinylidene Fluoride (PVDF), Polytetrafluoroethylene (PTFE), Silk, Ultra-high-molecular-weight polyethylene (UHMWPE) and stainless steel. Suture material structures may include, but are not limited to, monofilament, multifilament, braided and/or barb structures. Suture material may be natural or synthetic. Suture sizes may include, but are not limited to, a diameter in a range of 0.020 mm and 0.999 mm, or any value therebetween. Suturing techniques may include, but are not limited to, continuous suturing, interrupted suturing, deep suturing, buried suturing, purse-string suturing, and/or subcutaneous suturing.

Described herein are suture passer devices and methods.

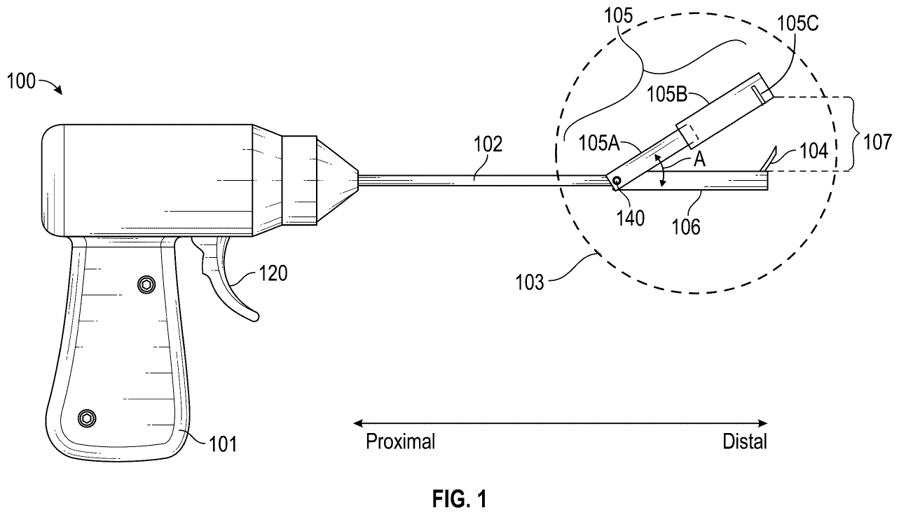

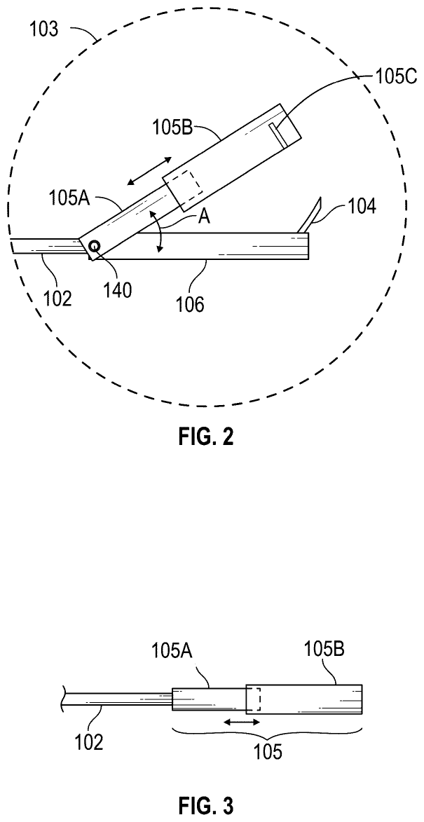

FIG. 1 shows a schematic side view of an embodiment of a suture passer device. FIG. 2 shows an enlarged schematic side view of an example of a suturing head of the suture passer device of FIG. 1.

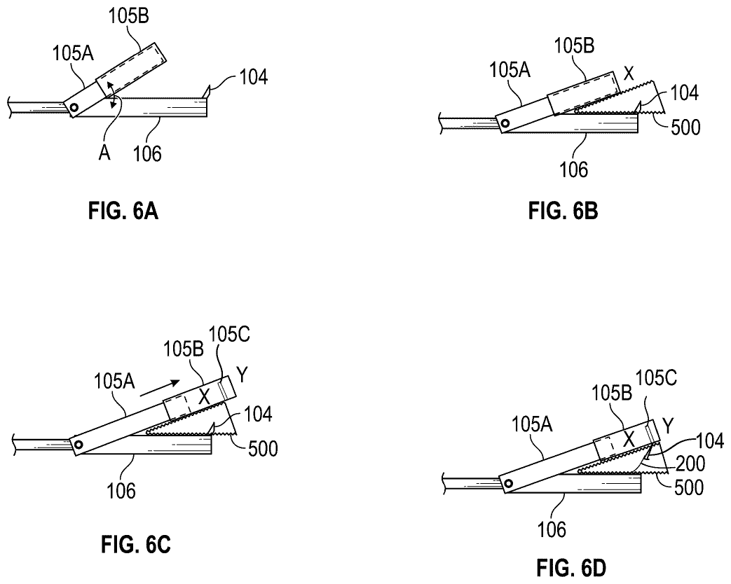

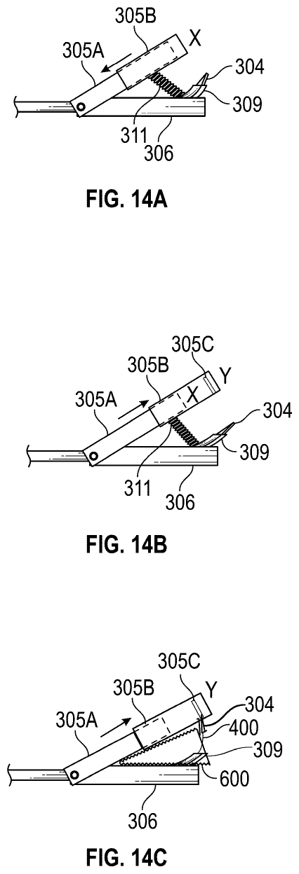

The suture passer device 100 is configured to pass a suture (see 200 in FIG. 6D, 400 in FIG. 14C) through a tissue to be sutured. In some embodiments, the suture passer device 100 may be handheld.

The suture passer device 100 may include a handle 101. The handle 101 may be adapted to be grasped by a hand of an operator. The handle 101, as depicted, is merely for convenience and it is understood that any suitable handle may be substituted for the handle 101 as disclosed herein. In some embodiments, the handle 101 may be an ergonomic handle.

The suture passer device 100 may include an elongate body 102. The elongate body 102 may extend from the handle 101. The elongate body 102 may be located at a distal end of the handle 101. As depicted, the elongate body 102 may be substantially planar or aligned with an upper surface of the handle 101. In some embodiments, the elongate body 102 may be angled respective to the upper surface of the handle 101. In some embodiments, the elongate body 102 may be angled down, up, to the left, or to the right with respect to the upper surface to any exemplary non-limiting angle, for example, 45°, 90°, or 120°. In some embodiments, the elongate body 102 may be curved in at least a section thereof at a proximal end, distal end, or any point therebetween. The curve may be directed to the right, left, up, or down. The elongate body 102, may be rigid or flexible.

In some embodiments, the elongate body 102 may include a housing (not shown) configured to house the needle 104. In some embodiments, the housing (not shown) may include a cannula extending along a length of the elongate body 102.

The suture passer device 100 may include a suturing head 103 at a distal end of the elongate body 102. The suturing head 103 may include a needle 104. The suturing head 103 may include a first jaw member 105. The suturing head 103 may include a second jaw member 106. The first jaw member 105 and the second jaw member 106 may be pivotably coupled together by a hinge mechanism 140.

The needle 104 may be configured to selectively engage a length of the suture (see 200 FIG. 6D, 400 FIG. 14C). In some embodiments, the needle 104 may be configured to extend from a distal end of the second jaw member 106 from a retracted position. In some embodiments, the needle 104 may selectively engage a length of the suture (see 200 in FIG. 6D, 400 in FIG. 14C) in a recess (not shown) thereof. In some embodiments, the needle 104 may penetrate the tissue to be sutured. In some embodiments, the needle 104 may extend toward an opening 105C of a distal part 105B of the first jaw member 105 to an extended position. In some embodiments, the needle 104 may direct a length of the suture (see 200 in FIG. 6D, 400 in FIG. 14C) to the opening 105C105C of the distal part 105B of the first jaw member 105. In some embodiments, the needle 104 may, in the extended position, release a length of the suture (see 200 in FIG. 6D, 400 in FIG. 14C) to the opening 105C105C of the distal part 105B of the first jaw member 105. In some embodiments, the needle 104 may retract to a proximal end of the second jaw member 106 to the retracted position.

In some embodiments, the recess 104A (not shown) of the needle 104 may be a slot, wedge or groove.

In some embodiments, the recess 104A (not shown) of the needle 104 may include a retainer region (not shown) configured to hold a length of the suture (see 200 in FIG. 6D, 400 in FIG. 14C) as the needle 104 extends from a retracted position to an extended position. In some embodiments, the retainer region (not shown) may include a lateral cut-out region of the needle 104.

The suture needle 104 may include different shapes, including but not limited to, straight, bi-curve, ¼^{th} circle, ⅛^{th} circle, ⅜^{th} circle, and/or ½ circle. The suture needle 104 may be of different types, including but not limited to, precision conventional cutting type, reverse cutting type, precision reverse cutting type, taper point type, taper cutting type, and/or spatula.

The suture needle 104 may be made of, but not limited to, stainless steel. In some embodiments, the suture needle 104 may be flexible.

In some embodiments, the first jaw member 105 may be an upper jaw member. The first jaw member 105 may be configured to pivot relative to the elongate body 102, e.g., pivoted in a plane as shown in the side view of FIGS. 1 and 2 by an angle A. In some embodiments, the first jaw member 105 may be configured to pivot in a range of 0 degrees to 90 degrees, or any value therebetween, relative to the elongate body 102. In some embodiments, the first jaw member 105 may be configured to pivot relative to the second jaw member 106. A pivot movement of the first jaw member 105 relative to the elongate body 102 and/or the second jaw member 106 may be configured to clamp tissue between the first jaw member 105 and the second jaw member 106 and/or release therefrom. In a clamping position, the first jaw member 105 may be substantially parallel to the second jaw member 106.

As depicted, the first jaw member 105 may be substantially planar or aligned with the elongate body 102. In some embodiments, the first jaw member 105 may be angled with respect to the elongate body 102. In some embodiments, the first jaw member 105 may be curved in at least a section thereof at a proximal end, distal end, or any point therebetween. In some embodiments, the curve may be directed toward the second jaw member 106.

In some embodiments, a control mechanism 120 may control an angle of pivot of the first jaw member 105 relative to the elongate body 102. In some embodiments, pushing the control mechanism 120 may cause the first jaw member 105 to pivot from an angle of 90 degrees relative to the elongate body 102 to a smaller angle. The smaller angle may include any value in a range of 0 degrees to 90 degrees relative to the elongate body 102.

In some embodiments, a locking mechanism (not shown) may be configured to lock or unlock the first jaw member 105 in a position desired by a user. In some embodiments, the locking mechanism (not shown) may be a part of the control mechanism 120.

The first jaw member 105 may include a proximal part 105A. The first jaw member 105 may include a distal part 105B. The distal part 105B may be movably coupled to the proximal part 105A. The distal part 105B may be movable along a length of the proximal part 105A. For example, the distal part 105B may linearly translate along a longitudinal direction of the first jaw member 105.

The proximal part 105A may be configured to allow a movement of the distal part 105B along a length thereof. The proximal part 105A is shown herein, for example, to be rectangular. The proximal part 105A may have different shapes and/or sizes, including, but not limited to, cylindrical, semi-circular, conical, pyramidal, trapezoidal, or a combination thereof.

In some embodiments, a movement of the distal part 105B along the proximal part 105A may be in a linear direction along a length of the proximal part 105A. FIG. 3 shows an enlarged schematic top view of an embodiment of the first jaw member 105 of the suturing head 103 of the suture passer device 100. FIG. 3 also shows the proximal part 105a and a linear direction of movement (e.g., linear translation) of the movable distal part 105B along a length of the proximal part 105A. The distal part 105B may be movable in a linear direction along substantially an entire length of the proximal part 105A. Accordingly, in the illustrated embodiments, the distal part 105B may slide along a longitudinal direction of the proximal part 105A. A user may manually grip and slide the distal part 105B relative to the proximal part 105A. In other embodiments, a control mechanism (not shown) may be engaged to impart translational movement to the distal part 105B.

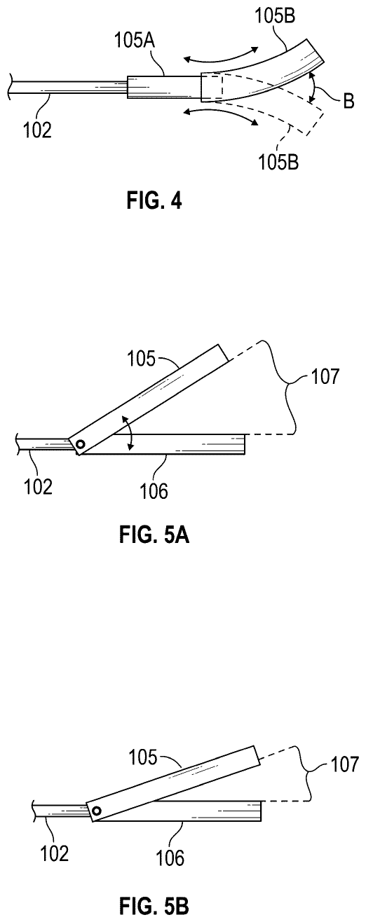

In some embodiments, a movement of the distal part 105B along the proximal part 105A may additionally or alternatively be in an angular direction relative to the proximal part 105A. FIG. 4 shows an enlarged schematic top view of an embodiment of the first jaw member 105 of the suturing head 103 of the suture passer device 100. FIG. 4 also shows the proximal part 105A and an angular direction of movement of the movable distal part 105B relative to the proximal part 105A. For example, as shown in FIG. 4, the distal part 105B can rotate in a plane as seen from a top view of the first jaw member 105 about an angle B. Movement of the distal part 105B can accordingly be rotated about the angle B which is a different angular orientation from the pivot angle A about which the jaw member(s) can be pivoted. An angular movement of the distal part 105B may be, but not limited to, in a range of 0 degrees to 20 degrees in either direction relative to the proximal part 105A.

As depicted, the distal part 105B may be substantially planar or aligned with the proximal part 105A. In some embodiments, the distal part 105B may be angled respective to the proximal part 105A. In some embodiments, the distal part 105B may be curved in at least a section thereof at a proximal end, distal end, or any point therebetween. In some embodiments, the curve may be directed toward the second jaw member 106.

A coupling mechanism of the distal part 105B to the proximal part 105A may include any mechanism that allows the distal part 105B to be movable along a length of the proximal part 105A. Such coupling mechanisms may include, but are not limited to, rollers, sliding tracks, rails, guides, and/or a combination thereof. In some embodiments, the coupling mechanism may be located on the proximal part 105A. In some embodiments, the coupling mechanism may be located on the movable distal part 105B.

In some embodiments, a control mechanism (not shown) may control an extent of movement of the distal part 105B relative to the proximal part 105A. The control mechanism may include any mechanism that causes movement of the distal part 105B relative to the proximal part 105A. Such mechanisms may include, but are not limited to, click wheels, levers, springs, and/or a combination thereof. In some embodiments, the control mechanism may be in proximity to the handle 101. In some embodiments, the control mechanism may be at a location to allow a user to use the control mechanism by a same hand holding the handle 101.

In some embodiments, a locking mechanism (not shown) may be configured to lock or unlock the distal part 105B in a position relative to the proximal part 105A as desired by a user. In some embodiments, the locking mechanism (not shown) may be a part of the control mechanism (not shown) that controls an extent of movement of the distal part 105B relative to the proximal part 105A.

The distal part 105B may include an opening 105C at a distal end thereof. In some embodiments, the opening 105C may be configured to capture a length of the suture (see 200 in FIG. 6D, 400 in FIG. 14C) from the needle 104. In some embodiments, the opening 105C may be configured to be a target guide for the needle 104. In some embodiments, the opening 105C may be configured to receive at least a part of the needle 104. In some embodiments, the opening 105C may include a recess (not shown) at the distal end of the distal part 105B. In some embodiments, the recess (not shown) may be a slot, wedge or groove.

In some embodiments, the opening 105C of the distal part 105B may include a suture capture member (not shown). In some embodiments, the suture capture member (not shown) may be configured to capture a length of the suture (see 200 in FIG. 6D, 400 in FIG. 14C) from the needle 104. In some embodiments, the suture capture member (not shown) may be configured to receive at least a part of the needle 104. In some embodiments, the suture capture member (not shown) may include a spring member, a proximal fixed end and a free distal end, and may be operable to be spring loaded by the needle 104 in the extended position. In some embodiments, a retraction of the needle 104 may release the suture capture member (not shown) to spring back and capture a length of the suture (see 200 in FIG. 6D, 400 in FIG. 14C) within the suture capture member (not shown).

The second jaw member 106 may be pivotally coupled to the first jaw member 105. In some embodiments, the second jaw member 106 may include a lower jaw member.

In some embodiments, the second jaw member 106 may be configured to pivot relative to the elongate body 102. In some embodiments, the second jaw member 106 may be configured to pivot in a range of 0 degrees to 90 degrees, or any value therebetween, relative to the elongate body 102. In some embodiments, the second jaw member 106 may be configured to pivot relative to the first jaw member 105. A pivot movement of the second jaw member 106 relative to the elongate body 102 and/or the first jaw member 105 may be configured to clamp tissue between the second jaw member 106 and the first jaw member 105 and/or release therefrom. In a clamping position, the second jaw member 106 may be substantially parallel to the first jaw member 105.

As depicted, the second jaw member 106 may be substantially planar or aligned with the elongate body 102. In some embodiments, the second jaw member 106 may be angled respective to the elongate body 102. In some embodiments, the second jaw member 106 may be curved in at least a section thereof at a proximal end, distal end, or any point therebetween. In some embodiments, the curve may be directed toward the first jaw member 105.

In some embodiments, a control mechanism (not shown) may control an angle of pivot of the second jaw member 106. In some embodiments, pushing the control mechanism (not shown) may cause the second jaw member 106 to pivot from an angle of 90 degrees relative to the elongate body 102 to a smaller angle. The smaller angle may include any value in a range of 0 degrees to 90 degrees relative to the elongate body 102.

In some embodiments, a locking mechanism (not shown) may be configured to lock or unlock the second jaw member 106 in a position desired by a user. In some embodiments, the locking mechanism (not shown) may be a part of the control mechanism (not shown) controlling an angle of pivot of the second jaw member 106.

The second jaw member 106 may be pivotally coupled to the first jaw member 105 to form a distal-facing mouth 107. The distal-facing mouth 107 may be configured to clamp a suture site 500 of the tissue in a closed configuration. FIGS. 5A and 5B show enlarged schematic side views of the distal-facing mouth 107 in an open configuration and closed configuration, respectively.

In some embodiments, at least one distal end of the first jaw member 105 and the second jaw member 106 may include features to enhance tissue clamping and/or suture passing stability. Such features may include, but are not limited to, serrated edges, fingers or teeth.

In some embodiments, the distal part 105B of the first jaw member 105 may be configured to be movable in a closed configuration of the distal-facing mouth 107. In some embodiments, the distal part 105B of the first jaw member 105 may be configured to be movable in an open configuration of the distal-facing mouth 107.

In some embodiments, the control mechanism 120 may be configured to adjust a position of the first jaw member 105 from an open configuration of the distal-facing mouth 107 to a closed configuration of the distal-facing mouth 107.

In some embodiments, the control mechanism (not shown) controlling the angle of pivot of the second jaw member 106 may be configured to adjust a position of the second jaw member 106 from an open configuration of the distal-facing mouth 107 to a closed configuration of the distal-facing mouth 107.

In some embodiments, the second jaw member 106 may be configured to allow movement therethrough of the needle 104 between a retracted position and an extended position. In some embodiments, the second jaw member 106 may include a channel (not shown) in at least a portion thereof. In some embodiments, a movement of the needle 104 between the retracted position and the extended position may be along a length of the channel (not shown) of the second jaw member 106.

In some embodiments, a control mechanism (not shown) may control a position of the needle 104 between a retracted position and an extended position, or any position therebetween. In some embodiments, pushing the control mechanism (not shown) may cause the needle 104 to extend from a retracted position to an extended position.

In some embodiments, a locking mechanism (not shown) may be configured to lock or unlock the needle 104 in a position desired by a user. In some embodiments, the locking mechanism (not shown) may be a part of the control mechanism (not shown) controlling a position of the needle 104.

In some embodiments, the control mechanism (not shown) may lock the needle 104 in the retracted position when the distal-facing mouth 107 is in an open configuration.

The second jaw member 106 may be configured at a distal end thereof to guide the needle 104 toward the opening 105C of the distal part 105B of the first jaw member 105. In some embodiments, the distal end of the second jaw member 106 may be configured to receive at least a part of the needle 104 as the needle extends from a retracted position.

In some embodiments, the distal end of the second jaw member 106 may include a recess (not shown) to selectively engage a length of the suture 200. In some embodiments, the recess (not shown) may be a slot, wedge or groove.

In some embodiments, the second jaw member 106 may include a first loading region (not shown) configured to hold a length of the suture 200. In some embodiments, the second jaw member 106 may include a second loading region (not shown) configured to hold at least one additional length of the suture 200.

In some embodiments, a locking mechanism (not shown) may be configured to lock or unlock the second jaw member 106 in a position desired by a user. In some embodiments, the locking mechanism (not shown) may be a part of the control mechanism (not shown) controlling an angle of pivot of the second jaw member 106.

FIGS. 6A-6D show enlarged schematic side views of an embodiment of the suturing head of the suture passer device of FIG. 1, shown in operation thereof.

FIG. 6A shows an embodiment of the suturing head 103, where the distal movable part 105B is slid inward relative to the proximal part 105A, the needle 104 is in a retracted position, and the distal-facing mouth 107 is in an open configuration.

FIG. 6B shows an embodiment where the distal-facing mouth 107 is in a closed configuration, where the first jaw member 105 and second jaw member 106 are in a clamping position, clamping the tissue site 500. FIG. 6B also shows the distal movable part 105B being slid inward relative to the proximal part 105A such that a distal end thereof is in a position to guide the needle 104 towards a suture site target X. FIG. 6B also shows the needle 104 in a retracted position.

FIG. 6C shows an embodiment where a suture site target Y is in a different position than suture site target X. Suture site target Y may be in a confined area not easily accessible and/or not visually accessible by current suture passer devices. In such cases, for example, the distal part 105B may be moved relative to the proximal part 105A such that a distal end of the distal part 105B is in a position to guide the needle 104 towards the suture site target Y. FIG. 6C shows the distal movable part 105B slid outward relative to the proximal part 105A such that a distal end thereof is in a position to guide the needle 104 towards the suture site target Y. Such movement of the distal part 105B relative to the proximal part 105A may be when the distal-facing mouth 107 is in a closed configuration, where the first jaw member 105 and second jaw member 106 are in a clamping position, clamping the tissue site 500. In some embodiments, such movement of the distal part 105B relative to the proximal part 105A may be when the distal-facing mouth 107 is in an open configuration.

FIG. 6D shows an embodiment where the distal movable part 105B is slid outward relative to the proximal part 105A such that a distal end thereof is in a position to guide the needle 104 towards the suture site target Y. FIG. 6D also shows an embodiment where the needle 104 is an extended position. FIG. 6D further shows an embodiment where the needle 104, and an engaged length of the suture 200, is guided to the suture site target Y.

In some embodiments, the handle 101 may include a handle assembly (not shown). In some embodiments, the handle assembly may house the control mechanism 120, the control mechanism (not shown) controlling an extent of movement of the distal part 105B relative to the proximal part 105A of the first jaw member 105, the control mechanism (not shown) controlling an angle of pivot of the second jaw member 106, and/or the control mechanism (not shown) controlling a position of the needle 104.

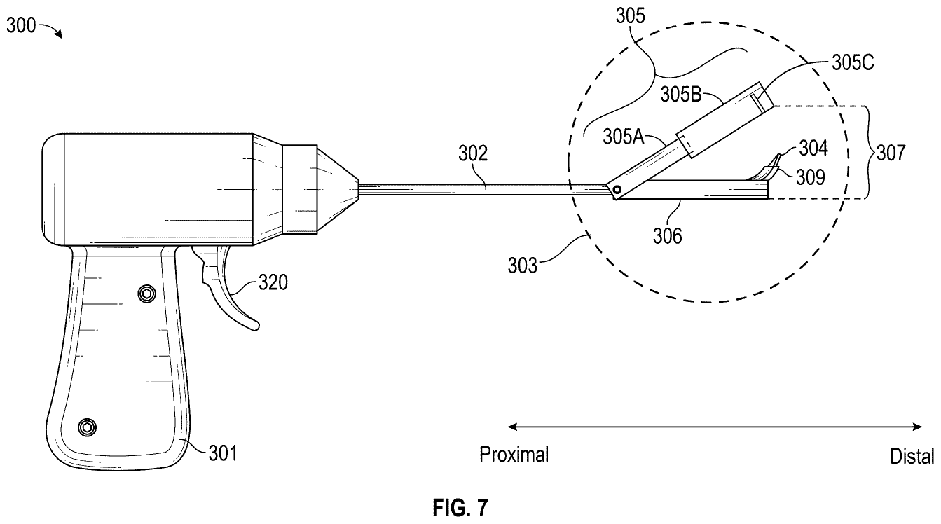

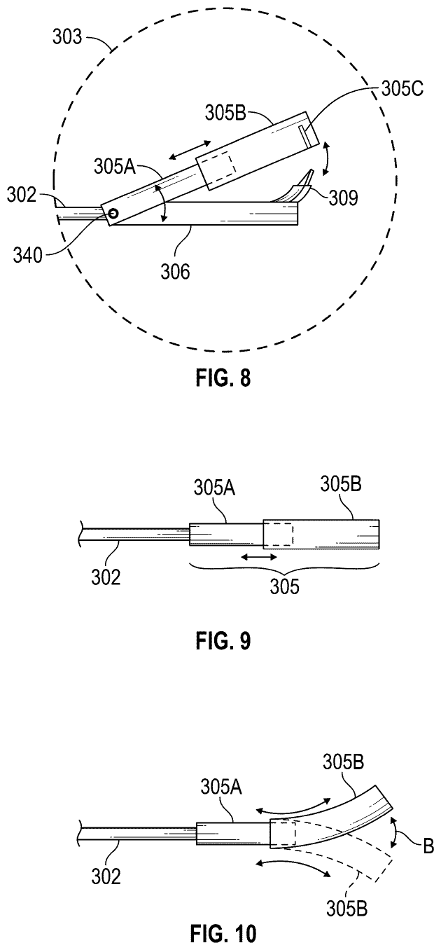

FIG. 7 shows a schematic side view of another example embodiment of a suture passer device. FIG. 8 shows an enlarged schematic side view of an embodiment of a suturing head of the suture passer device of FIG. 7.

In another aspect of the invention, a suture passer device 300 is configured to pass a suture (see 200 in FIG. 6D, 400 in FIG. 14C) through a tissue to be sutured. In some embodiments, the suture passer device 300 may be handheld.

The suture passer device 300 may include a handle 301. The handle 301 may be adapted to be grasped by a hand of an operator. The handle 301, as depicted, is merely for convenience and it is understood that any suitable handle may be substituted for the handle 301 as disclosed herein. In some embodiments, the handle 301 may be an ergonomic handle.

The suture passer device 300 may include an elongate body 302. The elongate body 302 may extend from the handle 301. The elongate body 302 may be located at a distal end of the handle 301. As depicted, the elongate body 302 may be substantially planar or aligned with an upper surface of the handle 301. In some embodiments, the elongate body 302 may be angled respective to the upper surface of the handle 301. In some embodiments, the elongate body 302 may be angled down, up, to the left, or to the right with respect to the upper surface to any exemplary non-limiting angle, for example, 45°, 90°, 120°, or the like. In some embodiments, the elongate body 302 may be curved in at least a section thereof at a proximal end, distal end, or any point therebetween. The curve may be directed to the right, left, upward, or downward. The elongate body 302, while shown to be rigid, may be flexible if desired.

In some embodiments, the elongate body 302 may include a housing (not shown) configured to house the needle 304. In some embodiments, the housing (not shown) may include a cannula extending along a length of the elongate body 302.

The suture passer device 300 may include a suturing head 303 at a distal end of the elongate body 302. The suturing head 303 may include a needle 304. The suturing head 303 may include a first jaw member 305. The suturing head 303 may include a second jaw member 306. The first jaw member 305 may be pivotably coupled to the second jaw member 306 by a hinge mechanism 340.

The needle 304 may be configured to selectively engage a length of the suture (see 200 in FIG. 6D, 400 in FIG. 14C). In some embodiments, the needle 304 may be configured to extend from a distal end of the second jaw member 306 from a retracted position. In some embodiments, the needle 304 may selectively engage a length of the suture 400 in a recess (not shown) thereof. In some embodiments, the needle 304 may penetrate the tissue to be sutured. In some embodiments, the needle 304 may extend toward an opening 305C of a distal part 305B of the first jaw member 305 to an extended position. In some embodiments, the needle 304 may direct a length of the suture (see 200 in FIG. 6D, 400 in FIG. 14C) to the opening 305C of the distal part 305B of the first jaw member 305. In some embodiments, the needle 304 may, in the extended position, release a length of the suture (see 200 in FIG. 6D, 400 in FIG. 14C) to the opening 305C of the distal part 305B of the first jaw member 305. In some embodiments, the needle 304 may retract to a proximal end of the second jaw member 306 to the retracted position.

In some embodiments, the needle 304 may include a recess (not shown). In some embodiments, the recess (not shown) of the needle 304 may be a slot, wedge or groove.

In some embodiments, the recess of the needle 304 may include a retainer region (not shown) configured to hold a length of the suture 400 as the needle 304 extends from a retracted position to an extended position. In some embodiments, the retainer region (not shown) may include a lateral cut-out region of the needle 304.

The suture needle 304 may include different shapes, including but not limited to, straight, bi-curve, ¼th circle, ⅛th circle, ⅜th circle, and/or ½ circle. The suture needle 304 may be of different types, including but not limited to, precision conventional cutting type, reverse cutting type, precision reverse cutting type, taper point type, taper cutting type, and/or spatula.

The suture needle 304 may be made of, but not limited to, stainless steel or other similar materials. In some embodiments, the suture needle 304 may be flexible.

In some embodiments, the first jaw member 305 may be an upper jaw member. The first jaw member 305 may be configured to pivot relative to the elongate body 302, e.g., pivoted in a plane as shown in the side view of FIGS. 1 and 2 (of a different aspect) by an angle A. In some embodiments, the first jaw member 305 may be configured to pivot in a range of 0 degrees to 90 degrees, or any value therebetween, relative to the elongate body 302. In some embodiments, the first jaw member 305 may be configured to pivot relative to the second jaw member 306. A pivot movement of the first jaw member 305 relative to the elongate body 302 and/or the second jaw member 306 may be configured to clamp tissue between the first jaw member 305 and the second jaw member 306 and/or release therefrom. In a clamping position, the first jaw member 305 may be substantially parallel to the second jaw member 306.

As depicted, the first jaw member 305 may be substantially planar or aligned with the elongate body 302. In some embodiments, the first jaw member 305 may be angled with respect to the elongate body 302. In some embodiments, the first jaw member 305 may be curved in at least a section thereof at a proximal end, distal end, or any point therebetween. In some embodiments, the curve may be directed toward the second jaw member 306.

In some embodiments, a control mechanism 320 may control an angle of pivot of the first jaw member 305 relative to the elongate body 302. In some embodiments, pushing the control mechanism 320 may cause the first jaw member 305 to pivot from an angle of 90 degrees relative to the elongate body 302 to a smaller angle. The smaller angle may include any value in a range of 0 degrees to 90 degrees relative to the elongate body 302.

In some embodiments, a locking mechanism (not shown) may be configured to lock or unlock the first jaw member 305 in a position desired by a user. In some embodiments, the locking mechanism (not shown) may be a part of the control mechanism 320.

The first jaw member 305 may include a proximal part 305A. The first jaw member 305 may include a distal part 305B. The distal part 305B may be movably coupled to the proximal part 305A. The distal part 305B may be movable along a length of the proximal part 305A. For example, the distal part 305B may linearly translate along a longitudinal direction of the first jaw member 305.

The proximal part 305A may be configured to allow a movement of the distal part 305B along a length thereof. The proximal part 305A is shown herein, for example, to be rectangular. The proximal part 305A may have different shapes and/or sizes, including, but not limited to, cylindrical, semi-circular, conical, pyramidal, trapezoidal, or a combination thereof.

In some embodiments, a movement of the distal part 305B along the proximal part 305A may be in a linear direction along a length of the proximal part 305A. FIG. 9 shows an enlarged schematic top view of an embodiment of the first jaw member 305 of the suturing head 303 of the suture passer device 300. FIG. 9 also shows the proximal part 305A and a linear direction of movement (e.g., linear translation) of the movable distal part 305B along a length of the proximal part 305A. The distal part 305B may be movable in a linear direction along substantially an entire length of the proximal part 305A. Accordingly, in the illustrated embodiments, the distal part 305B may slide along a longitudinal direction of the proximal part 305A. A user may manually grip and slide the distal part 305B relative to the proximal part 305A. In other embodiments, a control mechanism (not shown) may be engaged to impart translational movement to the distal part 305B.

In some embodiments, a movement of the distal part 305B along the proximal part 305A may additionally or alternatively be in an angular direction relative to the proximal part 305A. FIG. 10 shows an enlarged schematic top view of an embodiment of the first jaw member 305 of the suturing head 303 of the suture passer device 300. FIG. 10 also shows the proximal part 305A and an angular direction of movement of the movable distal part 305B relative to the proximal part 305A. For example, similar to that shown in FIG. 4 (showing a different aspect), the distal part 305B can rotate in a plane as seen from a top view of the first jaw member 305 about an angle B. Movement of the distal part 305B can accordingly be rotated about the angle B which is a different angular orientation from the pivot angle A about which the jaw member(s) can be pivoted. An angular movement of the distal part 305B may be, but not limited to, in a range of 0 degrees to 20 degrees in either direction relative to the proximal part 305A.

As depicted, the distal part 305B may be substantially planar or aligned with the proximal part 305A. In some embodiments, the distal part 305B may be angled respective to the proximal part 305A. In some embodiments, the distal part 305B may be curved in at least a section thereof at a proximal end, distal end, or any point therebetween. In some embodiments, the curve may be directed toward the second jaw member 306.

A coupling mechanism of the distal part 305B to the proximal part 305A may include any mechanism that allows the distal part 305B to be movable along a length of the proximal part 305A. Such mechanisms may include, but are not limited to, rollers, sliding tracks, rails, guides, and/or a combination thereof. In some embodiments, the coupling mechanism may be located on the proximal part 305A. In some embodiments, the coupling mechanism may be located on the movable distal part 305B.

In some embodiments, a control mechanism (not shown) may control an extent of movement of the distal part 305B relative to the proximal part 305A. The control mechanism may include any mechanism that causes movement of the distal part 305B relative to the proximal part 305A. Such mechanisms may include, but are not limited to, click wheels, levers, springs, and/or a combination thereof. In some embodiments, the control mechanism may be in proximity to the handle 301. In some embodiments, the control mechanism may be at a location to allow a user to use the control mechanism by a same hand holding the handle 301.

In some embodiments, a locking mechanism (not shown) may be configured to lock or unlock the distal part 305B in a position relative to the proximal part 305A as desired by a user. In some embodiments, the locking mechanism (not shown) may be a part of the control mechanism (not shown) that controls an extent of movement of the distal part 305B relative to the proximal part 305A.

The distal part 305B may include an opening 305C at a distal end thereof. In some embodiments, the opening 305C may be configured to capture a length of the suture (see 200 in FIG. 6D, 400 in FIG. 14C) from the needle 304. In some embodiments, the opening 305C may be configured to be a target guide for the needle 304. In some embodiments, the opening 305C may be configured to receive at least a part of the needle 304. In some embodiments, the opening 305C may include a recess (not shown) at the distal end of the distal part 305b. In some embodiments, the recess (not shown) may be a slot, wedge or groove.

In some embodiments, the opening 305C of the distal part 305B may include a suture capture member (not shown). In some embodiments, the suture capture member (not shown) may be configured to capture a length of the suture 400 from the needle 304. In some embodiments, the suture capture member (not shown) may be configured to receive at least a part of the needle 304. In some embodiments, the suture capture member (not shown) may include a spring member, a proximal fixed end and a free distal end, and may be operable to be spring loaded by the needle 304 in the extended position. In some embodiments, a retraction of the needle 304 may release the suture capture member (not shown) to spring back and capture a length of the suture 400 within the suture capture member (not shown).

The second jaw member 306 may be pivotally coupled to the first jaw member 305. In some embodiments, the second jaw member 306 may include a lower jaw member.

In some embodiments, the second jaw member 306 may be configured to pivot relative to the elongate body 302. In some embodiments, the second jaw member 306 may be configured to pivot in a range of 0 degrees to 90 degrees, or any value therebetween, relative to the elongate body 302. In some embodiments, the second jaw member 306 may be configured to pivot relative to the first jaw member 305. A pivot movement of the second jaw member 306 relative to the elongate body 302 and/or the first jaw member 305 may be configured to clamp tissue between the second jaw member 306 and the first jaw member 305 and/or release tissue therefrom. In a clamping position, the second jaw member 306 may be substantially parallel to the first jaw member 305.

As depicted, the second jaw member 306 may be substantially planar or aligned with the elongate body 302. In some embodiments, the second jaw member 306 may be angled respective to the elongate body 302. In some embodiments, the second jaw member 306 may be curved in at least a section thereof at a proximal end, distal end, or any point therebetween. In some embodiments, the curve may be directed toward the first jaw member 305.

In some embodiments, a control mechanism (not shown) may control an angle of pivot of the second jaw member 306. In some embodiments, pushing the control mechanism (not shown) may cause the second jaw member 306 to pivot from an angle of 90 degrees relative to the elongate body 302 to a smaller angle. The smaller angle may include any value in a range of 0 degrees to 90 degrees relative to the elongate body 302.

In some embodiments, a locking mechanism (not shown) may be configured to lock or unlock the second jaw member 306 in a position desired by a user. In some embodiments, the locking mechanism (not shown) may be a part of the control mechanism (not shown) controlling an angle of pivot of the second jaw member 306.

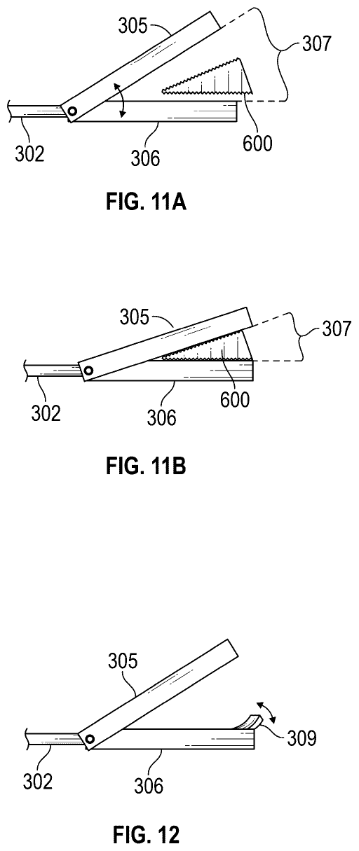

The second jaw member 306 may be pivotally coupled to the first jaw member 305 to form a distal-facing mouth 307. The distal-facing mouth 307 may be configured to clamp a suture site 600 of the tissue in a closed configuration. FIGS. 11A and 11B show enlarged schematic side views of the distal-facing mouth 307 in an open configuration and closed configuration, respectively.

In some embodiments, at least one distal end of the first jaw member 305 and the second jaw member 306 may include features to enhance tissue clamping and/or suture passing stability. Such features may include, but are not limited to, serrated edges, fingers or teeth.

In some embodiments, the distal part 305B of the first jaw member 305 may be configured to be movable in a closed configuration of the distal-facing mouth 307. In some embodiments, the distal part 305B of the first jaw member 305 may be configured to be movable in an open configuration of the distal-facing mouth 307.

In some embodiments, the control mechanism 320 may be configured to adjust a position of the first jaw member 305 from an open configuration of the distal-facing mouth 307 to a closed configuration of the distal-facing mouth 307.

In some embodiments, the control mechanism (not shown) controlling the angle of pivot of the second jaw member 306 may be configured to adjust a position of the second jaw member 306 from an open configuration of the distal-facing mouth 307 to a closed configuration of the distal-facing mouth 307.

In some embodiments, the second jaw member 306 may include a part 308, which may have different shapes and/or sizes, including, but not limited to, cylindrical, semi-circular, conical, pyramidal, trapezoidal, or a combination thereof.

In some embodiments, the part 308 may be configured to allow movement therethrough of the needle 304 between a retracted position and an extended position. In some embodiments, the part 308 may include a channel (not shown) in at least a portion thereof. In some embodiments, a movement of the needle 304 between the retracted position and the extended position may be along a length of the channel (not shown) of the part 308.

In some embodiments, a control mechanism (not shown) may control a position of the needle 304 between a retracted position and an extended position, or any position therebetween. In some embodiments, pushing the control mechanism (not shown) may cause the needle 304 to extend from a retracted position to an extended position.

In some embodiments, a locking mechanism (not shown) may be configured to lock or unlock the needle 304 in a position desired by a user. In some embodiments, the locking mechanism (not shown) may be a part of the control mechanism (not shown) controlling a position of the needle 304.

In some embodiments, the control mechanism (not shown) may lock the needle 304 in the retracted position when the distal-facing mouth 307 is in an open configuration.

The second jaw member 306 may include a ramp 309 arranged at a distal end thereof (shown in FIG. 7). The ramp 309 may be configured to guide the needle 304 toward the opening 305C of the distal part 305B of the first jaw member 305. The ramp 309 may be configured to receive at least a part of the needle 304 as the needle extends from a retracted position.

The ramp 309 may be at an angle relative to the second jaw member 306. In some embodiments, the ramp 309 may be curved in at least a section thereof at a proximal end, distal end, or any point therebetween. In some embodiments, the curve may be directed toward the first jaw member 305. In some embodiments, the ramp 309 may be semi-circular and curved in a direction of the first jaw member 305.

In some embodiments, the ramp 309 may be movably coupled to the second jaw member 306. The ramp 309 may be configured to be movable in a height direction relative to the second jaw member 306. FIG. 12 shows an enlarged schematic side view of an example embodiment of the second jaw member 306 of the suturing head 303 of the suture passer device 300. FIG. 12 also shows the ramp 309 and a direction of movement of the ramp 309 relative to the second jaw member 306.

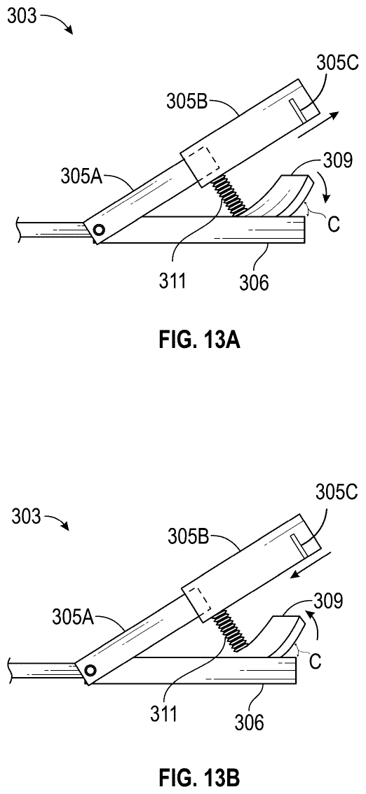

An angle C of the ramp 309 may be adjustable relative to the second jaw member 306. The ramp 309 may be configured so that an angle thereof relative to the second jaw member 306 may be in a range of 0 degrees to 90 degrees. FIGS. 13A and 13B are enlarged schematic side views of an example embodiment of the suturing head 303 of the suture passer device 300, showing changes in the angle C of the ramp 309 relative to the second jaw member 306 due to extension/retraction of the distal movable part 305B relative to the proximal part 305A of the first jaw member 305.

In some embodiments, the ramp 309 may be coupled via a coupling mechanism 311 to the distal movable part 305B of the first jaw member 305 so that a movement of the distal movable part 305B relative to the proximal part 305A automatically changes an angle C of the ramp 309 relative to the second jaw member 306. The change in angle C of the ramp 309 may be configured so that the ramp 309 always guides the needle 304 toward the opening 305C of the distal part 305B of the first jaw member 305.

An extension movement of the distal part 305B relative to the proximal part 305A of the first jaw member 305 may automatically decrease the angle C of the ramp 309 relative to the second jaw member 306. A retraction movement of the distal part 305B relative to the proximal part 305A of the first jaw member 305 may automatically increase the angle C of the ramp 309 relative to the second jaw member 306

In some embodiments, a movement of the distal movable part 305B relative to the proximal part 305A may automatically change an angle C of the ramp 309 relative to the second jaw member 306 in an open configuration of the distal-facing mouth 307.

In some embodiments, a movement of the distal movable part 305B relative to the proximal part 305A may automatically change an angle of the ramp 309 relative to the second jaw member 306 in a closed configuration of the distal-facing mouth 307.

In some embodiments, a control mechanism (not shown) may control an angle C of the ramp 309 relative to the second jaw member 306. In some embodiments, the control mechanism (not shown) may control the angle C of the ramp 309 independent of a movement of the distal part 305B relative to the proximal part 305A. In some embodiments, moving the control mechanism (not shown) in a specific direction may cause an angle C of the ramp 309 relative to the second jaw member 306 to decrease. In some embodiments, moving the control mechanism (not shown) in a direction opposite to the specific direction may cause an angle C of the ramp 309 relative to the second jaw member 306 to increase.

In some embodiments, the control mechanism (not shown) may control an angle C of the ramp 309 relative to the second jaw member 306 in an open configuration of the distal-facing mouth 307.

In some embodiments, the control mechanism (not shown) may control an angle C of the ramp 309 relative to the second jaw member 306 in a closed configuration of the distal-facing mouth 307.

The control mechanism (not shown) may include any mechanism that causes a change in an angle C of the ramp 309 relative to the second jaw member 306. Such mechanisms may include, but are not limited to, click wheels, levers, springs, and/or a combination thereof. In some embodiments, the control mechanism (not shown) may be in proximity to the handle 301. In some embodiments, the control mechanism may be at a location to allow a user to use the control mechanism (not shown) by a same hand holding the handle 301.

In some embodiments, a locking mechanism (not shown) may be configured to lock or unlock an angle C of the ramp 309 relative to the second jaw member 306 at a position desired by a user. In some embodiments, the locking mechanism (not shown) may be a part of the control mechanism (not shown) controlling an angle C of the ramp 309 relative to the second jaw member 306.

In some embodiments, the ramp 309 may include an opening (not shown) at a distal end thereof. In some embodiments, the opening (not shown) may be configured to receive at least a part of the needle 304. In some embodiments, the opening (not shown) may include a recess (not shown) at a distal end of the ramp 309 to selectively engage a length of the suture 400. In some embodiments, the recess (not shown) may be a slot, wedge or groove.

In some embodiments, the second jaw member 306 may include a first loading region (not shown) configured to hold a length of the suture 400. In some embodiments, the second jaw member 306 may include a second loading region (not shown) configured to hold at least one additional length of the suture 400.

FIGS. 14A-14C show enlarged schematic side views of an embodiment of the suturing head of the suture passer device of FIG. 7, shown in operation thereof.

FIG. 14A shows an embodiment of the suturing head 303, where the distal movable part 305B is slid inward relative to the proximal part 305A, the needle 304 is in a retracted position at a distal end of the ramp 309, and the distal-facing mouth 307 is in an open configuration. FIG. 14A also shows an example embodiment where the ramp 309 is in a position to guide the needle 304 to a suture site target X.

FIG. 14B shows an embodiment where a suture site target Y is in a different position than suture site target X. Suture site target Y may be in a confined area not easily accessible and/or not visually accessible by current suture passer devices. In such cases, for example, the distal part 305B may be moved relative to the proximal part 305A such that a distal end thereof is in a position to guide the needle 304 to the suture site target Y. FIG. 14B shows the distal movable part 305B slid outward relative to the proximal part 305A such that a distal end thereof is in a position to guide the needle 304 towards the suture site target Y. Such movement of the distal part 305B relative to the proximal part 305A may be when the distal-facing mouth 307 is in an open configuration.

In some embodiments, such movement of the distal part 305B relative to the proximal part 305A may be when the distal-facing mouth 307 is in a closed configuration, where the first jaw member 305 and second jaw member 306 are in a clamping position, clamping the tissue site 600.

FIG. 14B also shows an embodiment where ramp 309 may be coupled via a coupling mechanism 311 to the distal movable part 305B such that a movement of the distal movable part 305B relative to the proximal part 305A automatically changes an angle of the ramp 309 relative to the second jaw member 306 (shown in the enlarged view of FIG. 14A). In some embodiments, as described herein, an angle of the ramp 309 relative to the second jaw member 306 may be changed by a control mechanism. FIG. 14B shows an example embodiment where a change in the angle of the ramp 309 relative to the second jaw member 306 causes a distal end of the ramp 309 to be in a position to guide the needle 304 to the suture site target Y.

FIG. 14C shows an embodiment where the distal movable part 305B is slid outward relative to the proximal part 305A such that a distal end thereof is in a position to guide the needle 304 towards the suture site target Y. FIG. 14C also shows an embodiment where the needle 304 is an extended position. FIG. 14C further shows an embodiment where the angle C of the ramp 309 relative to the second jaw member 306 is such that a distal end of the ramp 309 is in a position to guide the needle 304 to the suture site target Y. FIG. 14C shows an embodiment where the needle 304, and an engaged length of the suture 400, is guided to the suture site target Y. In the embodiment of FIG. 14C, the distal-facing mouth 307 is in a closed configuration, where the first jaw member 305 and second jaw member 306 are in a clamping position, clamping the tissue site 600.

In some embodiments, the handle 301 may include a handle assembly (not shown). In some embodiments, the handle assembly may house the control mechanism 320, the control mechanism (not shown) controlling an extent of movement of the distal part 305b relative to the proximal part 305a of the first jaw member 305, the control mechanism (not shown) controlling an angle of pivot of the second jaw member 306, the control mechanism (not shown) controlling a position of the needle 304, and/or the control mechanism (not shown) controlling an angle of the ramp 309 relative to the second jaw member 306.

As seen from the examples above, the embodiments described herein provide advantages over current suture passer devices. The movability of the distal part(s) 105B or 305B relative to the proximal part(s) 105A or 305A, respectively, provide a user flexibility in guiding the needle(s) 104 or 304 with an engaged length of the suture(s) 200 or 400, respectively, to more than one suture site targets without having to change the distal-forming mouth(s) 107 or 307 from a closed configuration to an open configuration. These features of the disclosed embodiments provide the benefit that a clamp of the tissue site 500 or 600 by the upper jaw member(s) 105 or 305 and the lower jaw member(s) 106 or 306, respectively, need not be loosened/released to reach a new suture site target Y from a suture site target X. Thus, repeated puncturing of the tissue suture site 500 or 600 may be avoided by guiding the needle(s) 104 or 304 to a different suture site target Y by allowing adjustment(s) of the distal end(s) of the first jaw member(s) 105 or 305 without unclamping.

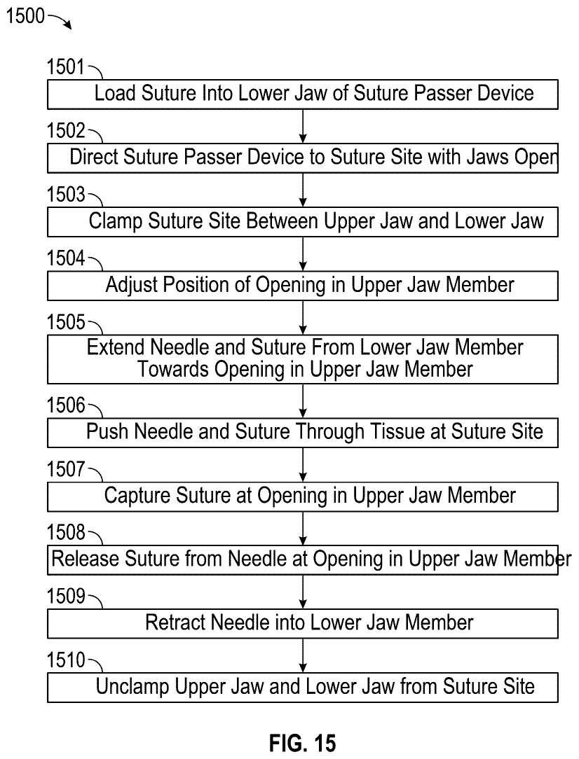

In another aspect of the invention, a method of passing suture through tissue may include providing a suture passer device 100 or 300 configured to pass a suture=(not shown) or 400 (not shown) through a tissue. FIG. 15 is a block diagram of an example method 1500 of passing a suture through a tissue using the suture passer device described herein. The method may include, at a step 1501, loading a length of the suture 200 (see 200 in FIG. 6D, 400 in FIG. 14C) into a respective second jaw member 106 or 306 of the suture passer device 100 or 300. The method may include, at a step 1502 directing the suture passer device 100 or 300 with a distal-facing mouth 107 or 307, respectively, in an open configuration to a site 500 or 600 to be sutured. The method may include, at a step 1503, adjusting a position of a first jaw member 105 or 305 of the suture passer device 100 or 300, respectively, and a position of the respective second jaw member 106 or 306 so that the respective distal-facing mouth 107 or 307 may be in a closed configuration clamping the tissue to be sutured. The method may include, at a step 1504, moving a movable distal part 105b or 305b of the first jaw member 105 or 305, respectively, along a length of a respective proximal part 105a or 305a of the first jaw member 105 or 305, respectively, to change a position of an opening 105C or 305C, respectively, at a respective distal part 105B or 305B of the first jaw member 105 or 305, respectively. The distal-facing mouth 107 or 307 may be in the closed configuration.

The method may include, at a step 1505, extending a needle 104 or 304 of the suture passer device 100 or 300, respectively from a retracted position. The needle 104 or 304 may be selectively engaged to a length of the suture 200 or 400. The method may include, at a step 1506, guiding the needle 104 or 304 toward the respective opening 105C or 305C at the distal part 105b or 305b, respectively, of the first jaw member 105 or 305. The method may include, at a step 1506, pushing the needle 104 or 304 through the tissue to be sutured. The needle 104 or 304 may guide a length of the suture 200 or 400 through the tissue toward the respective opening 105C or 305C at the distal part 105b or 305b, respectively, of the first jaw member 105 or 305. The method may include extending the needle 104 or 304 to the extended position.

The method may include, at a step 1507, capturing a length of the suture 200 or 400 at the opening 105C or 305C at the respective distal part 105B or 305B of the first jaw member 105 or 305. The method may include, at a step 1508, releasing a length of the suture 200 or 400 from the needle 104 or 304 to the respective opening 105C or 305C at the distal part 105B or 305B of the first jaw member 105 or 305. The method may include, at a step 1509, retracting the needle 104 or 304 to the retracted position. The method may include, at a step 1510, adjusting a position of the first jaw member 105 or 305 and a position of the second jaw member 106 or 306 so that the respective distal-facing mouth 107 or 307 may be in an open configuration. The method may include directing the suture passer device 100 or 400 away from the suture site 500 or 600. The opening 105C or 305C at the respective distal part 105B or 305B of the first jaw member 105 or 305 may be selectively engaged to a length of the suture 200 or 400.

In some embodiments, the method may include adjusting an angle of a ramp 309 at a distal end of the second jaw member 306 to guide the needle 304 toward the opening 305C at the distal part 305B of the first jaw member 305.

In another aspect of the invention, a method of passing suture through tissue may include providing a suture passer device 100 or 300 configured to pass a suture 200 or 400 through a tissue. The method may include loading a length of the suture 200 or 400 into a respective second jaw member 106 or 306 of the suture passer device 100 or 300. The method may include directing the suture passer device 100 or 300 with a distal-facing mouth 107 or 307, respectively, in an open configuration to a site 500 or 600 to be sutured. The method may include adjusting a position of a first jaw member 105 or 305 of the suture passer device 100 or 300, respectively, and a position of the respective second jaw member 106 or 306 so that the respective distal-facing mouth 107 or 307 may be in a closed configuration clamping the tissue to be sutured. The method may include moving a movable distal part 105B or 305B of the first jaw member 105 or 305, respectively, along a length of a respective proximal part 105A or 305A of the first jaw member 105 or 305, respectively, to change a position of an opening 105C or 305C, respectively, at a respective distal part 105B or 305B of the first jaw member 105 or 305, respectively. The distal-facing mouth 107 or 307 may be in an open configuration.

The method may include extending a needle 104 or 304 of the suture passer device 100 or 300, respectively from a retracted position. The needle 104 or 304 may be selectively engaged to a length of the suture 200 or 400. The method may include guiding the needle 104 or 304 toward the respective opening 105C or 305C at the distal part 105B or 305B, respectively, of the first jaw member 105 or 305. The method may include pushing the needle 104 or 304 through the tissue to be sutured. The needle 104 or 304 may guide a length of the suture 200 or 400 through the tissue toward the respective opening 105C or 305C at the distal part 105B or 305B, respectively, of the first jaw member 105 or 305. The method may include extending the needle 104 or 304 to the extended position.

The method may include capturing a length of the suture 200 or 400 at the opening 105C or 305C at the respective distal part 105B or 305B of the first jaw member 105 or 305. The method may include releasing a length of the suture 200 or 400 from the needle 104 or 304 to the respective opening 105C or 305C at the distal part 105B or 305B of the first jaw member 105 or 305. The method may include retracting the needle 104 or 304 to the retracted position. The method may include adjusting a position of the first jaw member 105 or 305 and a position of the second jaw member 106 or 306 so that the respective distal-facing mouth 107 or 307 may be in an open configuration. The method may include directing the suture passer device 100 or 400 away from the suture site 500 or 600. The opening 105C or 305C at the respective distal part 105B or 305B of the first jaw member 105 or 305 may be selectively engaged to a length of the suture 200 or 400.

In some embodiments, the method may include adjusting an angle of a ramp 309 at a distal end of the second jaw member 306 to guide the needle 304 toward the opening 305C at the distal part 305B of the first jaw member 305.

Various modifications to the embodiments described in this disclosure will be readily apparent to those skilled in the art, and the generic principles defined herein may be applied to other embodiments without departing from the spirit or scope of this disclosure. Thus, the disclosure is not intended to be limited to the embodiments discussed herein but is to be accorded the widest scope consistent with the claims, the principles and the novel features disclosed herein. The word “example” is used exclusively herein to mean “serving as an example, instance, or illustration.” Any embodiment described herein as “example” is not necessarily to be construed as preferred or advantageous over other embodiments, unless otherwise stated.

Certain features that are described in this specification in the context of separate embodiments also may be embodied in combination in a single embodiment. Conversely, various features that are described in the context of a single embodiment also may be embodied in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the order shown or in sequential order, or that all illustrated operations be performed, to achieve desirable results. Additionally, other embodiments are within the scope of the following claims. In some cases, the actions recited in the claims may be performed in a different order and still achieve desirable results.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

What is claimed is:

1. A suture passer device configured to pass a suture through a tissue, the device comprising a handle, an elongate body extending from the handle, and a suturing head at a distal end of the elongate body, the suturing head comprising:
a first jaw member comprising a proximal part and a distal part movably coupled to the proximal part and movable along a length of the proximal part, the distal part comprising an opening at a distal end thereof, the opening configured to capture a suture from a needle; and
a second jaw member pivotally coupled to the first jaw member to form a distal-facing mouth, the distal-facing mouth configured to clamp a suture site of the tissue in a closed configuration, the second jaw member configured to allow movement of the needle between a retracted position and an extended position, the second jaw member having a distal end configured to guide the needle toward the opening of the distal part of the first jaw member.

2. The suture passer device of claim 1, wherein the second jaw member comprises a ramp at a distal portion of the second jaw member, and wherein the ramp is configured to guide the needle toward the opening of the distal part of the first jaw member.

3. The suture passer device of claim 2, wherein an angle of the ramp is adjustable.

4. The suture passer device of claim 1, wherein the second jaw member comprises a channel so that the movement of the needle between the retracted position and the extended position is along a length of the channel.

5. The suture passer device of claim 1, wherein the first jaw member and/or the second jaw member is configured to pivot relative to the elongate body.

6. The suture passer device of claim 1, wherein the second jaw member is curved and has a radius of curvature.

7. The suture passer device of claim 1, wherein the second jaw member further comprises a recess at the distal end configured to selectively engage a length of the suture.

8. The suture passer device of claim 1, wherein the second jaw member further comprises a first loading region configured to hold a length of the suture.

9. The suture passer device of claim 2, wherein the ramp comprises a recess to selectively engage a length of the suture.

10. The suture passer device of claim 2, wherein the ramp is semi-circular.

11. The suture passer device of claim 1, wherein the suture passer device is handheld.

12. The suture passer device of claim 1, wherein the opening of the first jaw member is configured to receive the needle.

13. The suture passer device of claim 1, wherein the distal part of the first jaw member is movable in a linear direction along a length of the proximal part.

14. The suture passer device of claim 1, wherein the distal part of the first jaw member is movable in an angular direction relative to a length of the proximal part.

15. The suture passer device of claim 1, wherein the distal part of the first jaw member is configured to be movable in a closed configuration of the distal-facing mouth.

16. The suture passer device of claim 1, wherein the distal part of the first jaw member is configured to be movable in an open configuration of the distal facing mouth.

17. The suture passer device of claim 1, wherein the elongate body comprises a housing configured to house the needle.

18. A method of passing a suture through tissue, the method comprising:
engaging a tissue with a suture device comprising a lower jaw member and an upper jaw member, the upper jaw member being pivotable relative to the lower jaw member, the upper jaw member comprising a proximal part and a distal part movable relative to the proximal part;
translating the distal part relative to the proximal part along a longitudinal direction of the upper jaw member; and
directing a suture through an opening in the upper jaw member to close the tissue.

19. The method of claim 18, further comprising directing the suture along the lower jaw member to engage a ramp, the ramp directing the suture into the tissue and toward the opening.

20. The method of claim 18, further comprising adjusting an angle of the ramp relative to the lower jaw member.
